(19) United States
(12) Patent Application Publication
(10) Pub. No.: US 2025/0339152 A1
(43) Pub. Date: Nov. 6, 2025

(54) TPU SUBSTRATE FORMING AN EMBOLIZATION DEVICE FOR TREATING DEFECT VASCULAR VESSEL

(71) Applicant: DINHTEC, INC., Irvine, CA (US)

(72) Inventors: Linh Dinh, Irvine, CA (US); Chau B. Pham, Irvine, CA (US)

(73) Assignee: DINHTEC, INC., Irvine, CA (US)

(21) Appl. No.: 18/655,719

(22) Filed: May 6, 2024

(51) Int. Cl. A61B 17/12; A61B 17/00

(52) U.S. Cl. CPC A61B 17/12118; A61B 17/00234; A61B 17/12031; A61B 17/12145; A61B 17/1215; A61B 2017/00292; A61B 2017/00477; A61B 2017/00836; A61B 2017/0084; A61B 2017/00862; A61B 2017/00867; A61B 2017/00893; A61B 2017/00938; A61B 2017/00946; A61B 2017/1205

(57) ABSTRACT

The present invention compromises a cerebral aneurysm device designed for insertion in various cerebral aneurysm types and block blood flow into a cerebral aneurysm to prevent it from rupturing.

### CROSS-REFERENCE TO RELATED APPLICATIONS

N/A

### FIELD OF THE INVENTION

The invention relates to a device used in the endovascular procedure, in particular, a minimally invasive device to treat a cerebral aneurysm. Its purpose is to block the blood flow into the aneurysm which is a weakened area in the wall of an artery.

### BACKGROUND

A cerebral aneurysm is a weak or thin spot on an artery in the brain that bulges out and fills with blood, see FIG. 1. The bulging aneurysm may burst or rupture which spills blood into the surrounding tissue (called a hemorrhage). A ruptured aneurysm can cause serious health problems such as hemorrhagic stroke, brain damage, coma, and even death.

### Current Cerebral Aneurysm Treatments:

### 1. Endovascular Coiling:

For the unruptured aneurysm, that can be treated by surgical clipping and endovascular coiling. Endovascular coiling is a form of embolization to block blood flow into an aneurysm to keep it from rupturing, see FIG. 2. These coils are made of soft platinum metal wire that wound and shaped set as a long coil. The goal of the treatment is to pack the aneurysm tightly with as much coils as possible to cut off the flow of the blood to the wall of the budging artery to prevent it from rupturing. In the normal procedure, it may take a lot of coils and time to pack the average aneurysm.

The Endovascular Coiling requires a lot of coils to pack the aneurysm which yield high cost and long procedural time (potential further complication). For patients with wide neck aneurysm, the procedure requires additional intervention which is another extra cost and device/time.

### 2. Web Embolization System:

Medical innovation invention has been progressing. More new devices have been introduced to the market to treat the same aneurysm. The device called WEB Embolization System, which is a self-expanding Nitinol braided meshes shaped set as a ball or basket, see FIG. 3. Its purpose is to disrupt to blood flow entering the aneurysm and promoting clot (thrombosis). However, this device is also required some coils to pack the aneurysm. It requires less coil as compared to the Endovascular coiling itself. The device is also patient selective.

Even though WEB Embolization system doesn't need as many coils as Endovascular coiling device, WEB still needs coils to fill out the basket. Besides that, WEB doesn't have the answer for the wide neck aneurysm. It requires a bridge stent to close the neck of the aneurysm.

### 3. Extra Intervention for the Treatment:

In special cases of the wide neck aneurysm, both the Coiling and the WEB devices need another intervention-device, such as a bridge stent to mitigate the issue of coils or WEB herniate into the main vessel, see FIG. 4.

Both Coiling & Web treatments are known in the industry but these treatments could be further improved. Therefore, what is needed is a new cerebral aneurysm device that will minimize the procedural time and reduce the number of coils filling to improve procedure treatment and cost effective.

### BRIEF SUMMARY OF THE INVENTION

The present invention is a cerebral aneurysm device that minimizes the issue of using so many individual coils to pack the aneurysm. The cerebral aneurysm device disclosed is a multiple component system that is designed to improve the procedural time and intervention step.

### BRIEF DESCRIPTION OF THE DRAWINGS

FIG. 1 shows cerebral aneurysm on an artery in the brain that bulges out and fills with blood.

FIG. 2 shows a prior art device using endovascular coiling to block blood flow into an aneurysm to keep it from rupturing.

FIG. 3 show a prior art device using WEB Embolization System having a self-expanding Nitinol braided mesh to block blood flow into an aneurysm to keep it from rupturing.

FIG. 4 shows a prior art device using endovascular coiling to block blood flow into an aneurysm to keep it from rupturing and a bridge stent to mitigate the issue of coils herniate into the artery.

FIG. 5 shows one embodiment of a cerebral aneurysm device designed to fit in a narrow neck cerebral aneurysm.

FIG. 6 shows one embodiment of a cerebral aneurysm device designed to fit in a narrow to medium neck cerebral aneurysm.

FIG. 7 shows one embodiment of a cerebral aneurysm device designed to fit in a wide neck cerebral aneurysm.

FIGS. 8A-8D show one embodiment of a cerebral aneurysm device designed to prevent blood from entering a narrow neck cerebral aneurysm.

FIGS. 9A, 9B show one embodiment of a cerebral aneurysm device designed to prevent blood from entering a narrow to medium neck cerebral aneurysm.

FIGS. 10A, 10B show one embodiment of a cerebral aneurysm device designed to prevent blood from entering a wide neck cerebral aneurysm.

FIGS. 11A, 11B show one embodiment of a cerebral aneurysm device designed to prevent blood from entering a wide neck cerebral aneurysm.

FIGS. 12A, 12B show one embodiment of a cerebral aneurysm device designed to prevent blood from entering a narrow neck cerebral aneurysm.

FIGS. 13A, 13B show one embodiment of a cerebral aneurysm device designed to prevent blood from entering a wide neck cerebral aneurysm.

FIGS. 14A, 14B show one embodiment of a cerebral aneurysm device designed to prevent blood from entering a narrow to medium neck cerebral aneurysm.

FIGS. 15A, 15B show one embodiment of a cerebral aneurysm device designed to prevent blood from entering a wide neck cerebral aneurysm.

### DETAILED DESCRIPTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

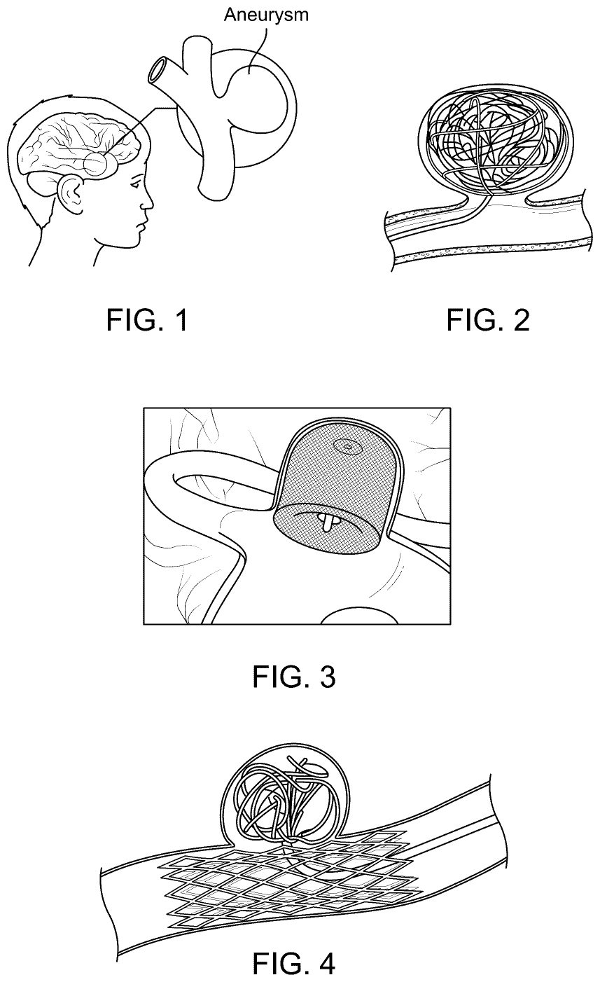

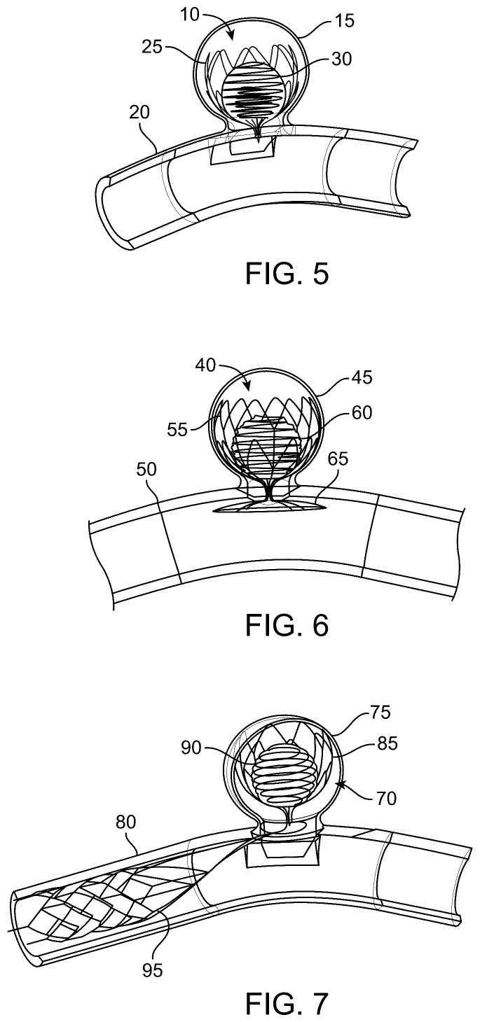

FIGS. 5-7 show an overview of cerebral aneurysm devices designed for insertion in various cerebral aneurysm types and block the blood flow into a cerebral aneurysm to prevent it from rupturing. The different cerebral aneurysm types include a narrow neck aneurysm (FIG. 1), a narrow to medium neck aneurysm (FIG. 2), or a wide neck aneurysm (FIG. 3).

FIG. 5 shows one embodiment of a cerebral aneurysm device 10 designed to fit in a narrow neck cerebral aneurysm 15 space on a cerebral artery 20. The cerebral aneurysm device 10 has multiple components including a frame 25 that has a basket or half spherical shape and a filler 30 positioned within the frame.

FIG. 6 shows one embodiment of a cerebral aneurysm device 40 designed to fit in a narrow to medium neck cerebral aneurysm 45 space on a cerebral artery 50. The cerebral aneurysm device 40 has multiple components including a frame 55 that has a basket or half spherical shape with a flared-out base, a filler 60 positioned within the frame, and a disk 65 coupled with the flared-out base. The half spherical shaped frame 55 is positioned within the cerebral aneurysm and the flared-out base with the disk 65 positioned in the cerebral artery 20 to cover the neck of the aneurysm.

FIG. 7 shows one embodiment of a cerebral aneurysm device 70 designed to fit a wide neck cerebral aneurysm 75 space on a cerebral artery 80. The cerebral aneurysm device 70 has multiple components including a frame 85 that has a basket or half spherical shape and a stent base 95, a filler 90 positioned within the frame. The stent base 95 is used to stabilize cerebral aneurysm device 70 in the aneurysm and anchor the cerebral aneurysm device 70 so it cannot herniate into the vessel.

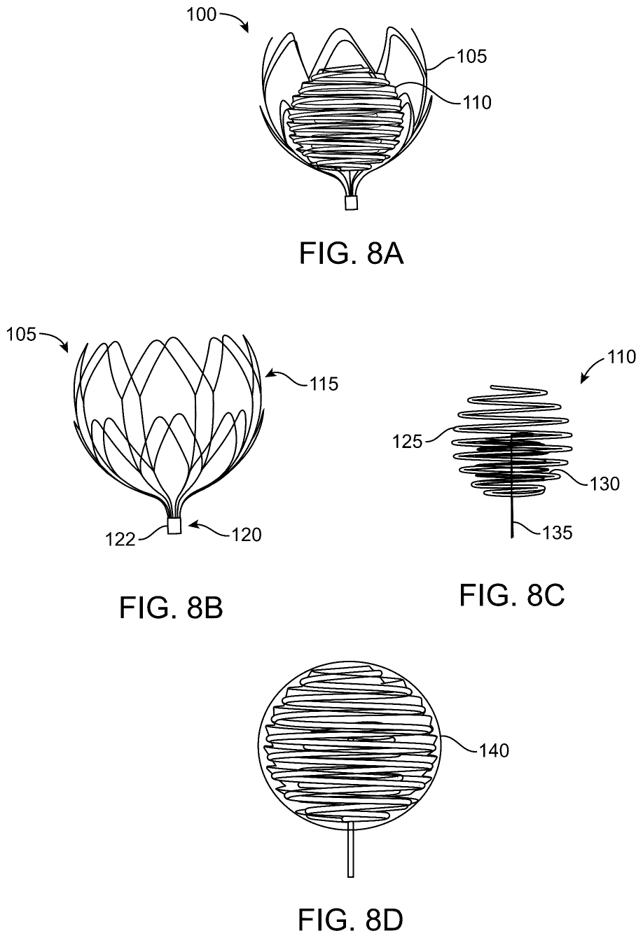

FIGS. 8A-8D show one embodiment of a cerebral aneurysm device 100 designed to prevent blood from entering a narrow neck cerebral aneurysm. The cerebral aneurysm device 100 is a multiple component device having a frame 105 with a central opening and a filler 110 with a coating or film positioned in the central opening. The cerebral aneurysm device 100 may be collapsed or compressed into a delivery configuration sized to fit within a lumen of a delivery catheter and delivered to the cerebral aneurysm. The cerebral aneurysm device 100 exits the lumen and self-expands to prevent blood from entering a narrow neck cerebral aneurysm.

The frame 105 has a basket or half spherical shape 115 forming a central opening with a closed end 120. The closed end includes a hub or band 122 to keep the closed end from opening during compression and expansion. In the embodiment shown, the basket or half spherical shape 115 is made of petal shaped arms extending upward. In other embodiments, the basket or half spherical shape 115 is made of finger like arms. In one embodiment, the frame 105 is made of a self-expanding material, such as Nitinol, that is laser cut and pre-shaped in the basket or half spherical shape. In other embodiments, the frame 105 is made of a self-expanding wire material, such as Nitinol wire, that is shaped into the basket or half spherical shape and joined at the closed end 120.

The filler 110 may be made of a self-expanding wire material, such as Nitinol wire, that is pre-shaped. The filler 110 includes an outer spherical-shaped coil portion 125 and an inner spherical-shaped coiled portion 130 with a segment 135 configured to attach with the closed end 120 of the frame 105. In some embodiments, the closed end includes a hub or band 122 to keep the closed end from opening during expansion. The filler coil portion 125 may have variant pitch and amplitude for different purposes. A coating or film 140 covers spherical-shaped coil portion 125 and seals the filler 110. The coating or film 140 may be made from a polyurethane, ePTFE and/or other collagen film, and may have various functions, such as:
The coating or film can function as a filler (as compared to the coil) to block or deflect the blood flow into the aneurysm to protect the weakened wall of the artery.
The coating or film can be a platform as a drug carrier that can have a two phases release, one side promoting the endothelial cell and other side can promoting thromboses.

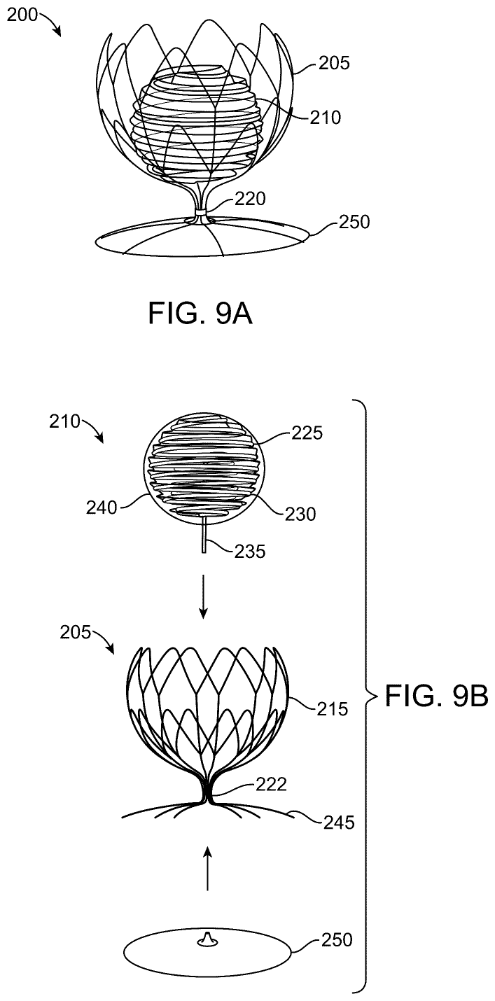

FIGS. 9A, 9B show one embodiment of a cerebral aneurysm device 200 designed to prevent blood from entering a narrow to medium neck cerebral aneurysm. The cerebral aneurysm device 200 is a multiple component device including a frame 205 having a central opening with a closed end 220 and a flared-out base, a filler 210 positioned within the central opening, and a disk 250 coupled to the flared-out base. The cerebral aneurysm device 200 may be collapsed or compressed into a delivery configuration sized to fit within a lumen of a delivery catheter and delivered to the narrow to medium neck cerebral aneurysm. The cerebral aneurysm device 200 exits the lumen and self-expands, with the pre-shaped basket or half spherical shaped frame 205 and filler 210 self-expanding within the aneurysm to fill the aneurysm, and the flared-out base and disk self-expanding in the artery to cover the narrow to medium neck, blocking the flow of blood into the aneurysm.

The frame 205 has a basket or half spherical shape 215 forming a central opening with a closed end 220, and a flared base 245. The closed end includes a hub or band 222 to keep the closed end from opening during compression and expansion. The frame 205 may be made of a self-expanding material, such as Nitinol, that is laser cut and pre-shaped in the basket or half spherical shape and flared out base. The hub or band 222 is coupled to the closed end 220 to keep the closed end from opening during self-expansion and the flared-out base 245 extends outwardly from the hub or band 222.

The filler 210 may be the same as filler 110, having an outer spherical-shaped coil portion 225 and an inner coiled portion 230 with a segment 235 configured to attach with the closed end 220 of the frame 205. The inner and outer filler coil portions 225, 230 may have variant pitch and amplitude for different purposes, such as the inner coil portion having a first pitch and a first amplitude, and the outer coil portion having a second pitch and a second amplitude. A coating or film 240 covers spherical-shaped coil portion 225 and seals the filler 210. The coating or film 240 may be made from a polyurethane and/or ePTFE film. The disk 250 is designed to be a barrier reinforced by the flared base 245. The disk 250 may be made from a polyurethane and/or ePTFE film. The disk 250 may also include a coating or film can be a platform as a drug carrier promoting the endothelial cell.

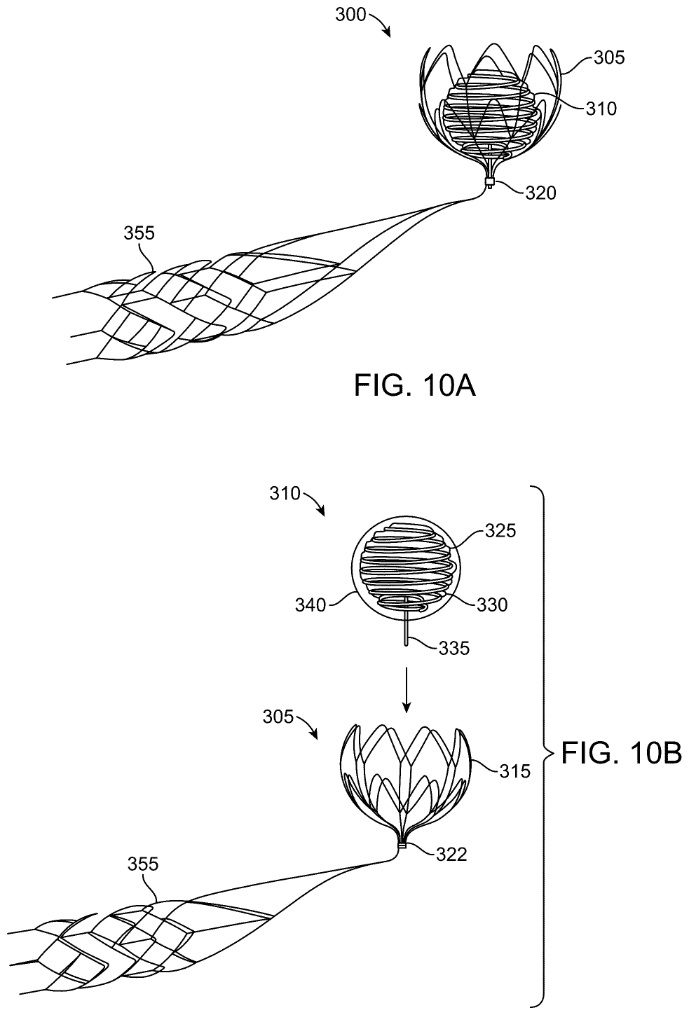

FIGS. 10A, 10B show one embodiment of a cerebral aneurysm device 300 designed to prevent blood from entering a wide neck cerebral aneurysm. The cerebral aneurysm device 300 is a multiple component device including a frame 305 having a central opening with a closed end and a stent base, and a filler 310 positioned within the central opening. The cerebral aneurysm device 300 may be collapsed or compressed into a delivery configuration sized to fit within a lumen of a delivery catheter and delivered to the wide neck cerebral aneurysm. The cerebral aneurysm device 300 exits the lumen and self-expands, with the pre-shaped basket or half spherical shape frame 305 and filler 310 self-expanding within the aneurysm to fill the aneurysm, and the stent base configured to expand within the artery to anchor the cerebral aneurysm device 300.

The frame 305 has a basket or half spherical shape 315 forming a central opening with a closed end 320 and a stent base 355. The closed end includes a hub or band 322 to keep the closed end from opening during compression and expansion. The frame 305 may be made of a self-expanding material, such as Nitinol, that is laser cut and pre-shaped in the basket or half spherical shape and the stent base shape. The hub or band 322 is coupled to the closed end 320 to keep the closed end from opening during self-expansion and the stent base 355 extends outwardly from the hub or band 322 in the artery. The stent base 355 is used to stabilize cerebral aneurysm device 300 in the aneurysm and anchor the cerebral aneurysm device 300 so it cannot herniate into the vessel.

The filler 310 may be the same as filler 110, having an outer spherical-shaped coil portion 325 and an inner coiled portion 330 with a segment 335 configured to attach with the closed end 320 of the frame 305. The filler coil portion 325 may have variant pitch and amplitude for different purposes. A coating or film 340 covers spherical-shaped coil portion 325 and seals the filler 310. The coating or film 340 may be made from a polyurethane and/or ePTFE film.

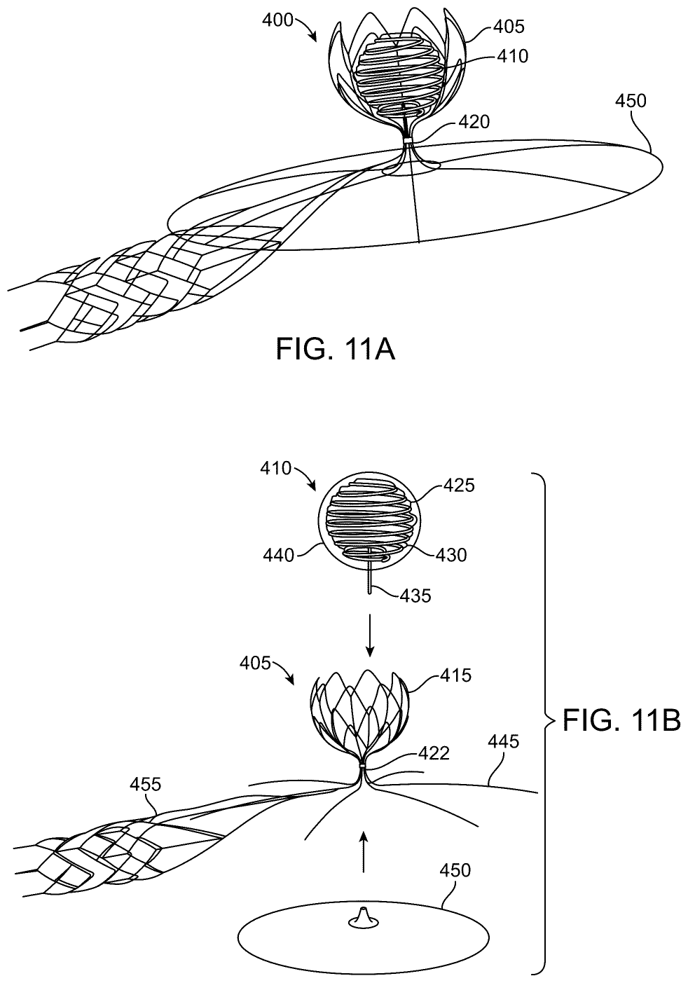

FIGS. 11A, 11B show one embodiment of a cerebral aneurysm device 400 designed to prevent blood from entering a wide neck cerebral aneurysm. The cerebral aneurysm device 400 is similar to cerebral aneurysm device 300 with the addition of a disk configured to cover the wide neck. The cerebral aneurysm device 400 is a multiple component device including a frame 405 having a central opening with a closed end, a flared-out base and a stent base, a filler 410 positioned within the central opening, and a disk 450 coupled to the flared-out base. The cerebral aneurysm device 400 may be collapsed or compressed into a delivery configuration sized to fit within a lumen of a delivery catheter and delivered to the wide neck cerebral aneurysm. The cerebral aneurysm device 400 exits the lumen and self-expands, with the pre-shaped basket or half spherical shaped frame 405 and filler 410 self-expanding within the aneurysm to fill the aneurysm, the flared-out frame with disk 450 self-expanding in the artery to cover the wide neck blocking the flow of blood into the aneurysm, and the stent base configured to self-expand within the artery to anchor the cerebral aneurysm device 400.

The frame 405 has a basket or half spherical shape 415 forming a central opening with a closed end 420, a flared base 445 and a stent base 455. The closed end includes a hub or band 422 to keep the closed end from opening during compression and expansion. The frame 405 may be made of a self-expanding material, such as Nitinol, that is laser cut and pre-shaped in the basket or half spherical shape, the flare out base, and the stent base shape. The hub or band 422 is coupled to the closed end 420 to keep the closed end from opening during self-expansion and the flared out base 445 extends outwardly from the hub or band 422.

The filler 410 may be the same as filler 110, having an outer spherical-shaped coil portion 425 and an inner coiled portion 430 with a segment 435 configured to attach with the closed end 420 of the frame 405. The filler coil portion 425 may have variant pitch and amplitude for different purposes. A coating or film 440 covers spherical-shaped coil portion 425 and seals the filler 410. The coating or film 440 may be made from a polyurethane and/or ePTFE film. The disk 450 may be made from a polyurethane and/or ePTFE film. The disk 450 is designed to be a barrier reinforced by the flared base 445. The disk 450 may also include a coating or film that can be a platform as a drug carrier promoting the endothelial cell.

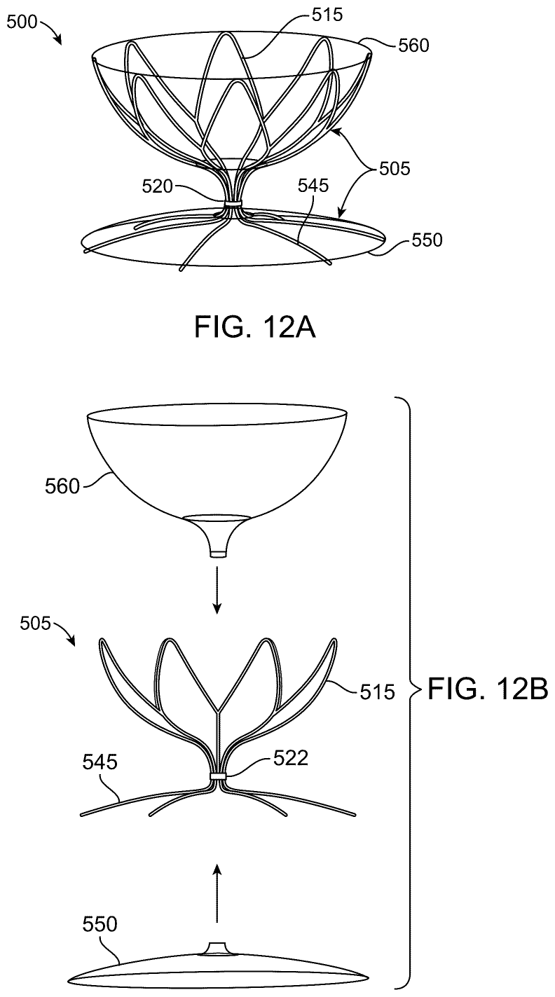

FIGS. 12A, 12B show one embodiment of a cerebral aneurysm device 500 designed to prevent blood from entering a narrow to medium neck cerebral aneurysm. The cerebral aneurysm device 500 is a multiple component device including a frame 505 having basket or half spherical shape 515 and a flared base 545, a covering 560 coupled to basket or half spherical shape 515, and a disk 550 coupled to the flared-out base 545. The cerebral aneurysm device 500 may be collapsed or compressed into a delivery configuration sized to fit within a lumen of a delivery catheter and delivered to the narrow to medium neck cerebral aneurysm. The cerebral aneurysm device 500 exists the lumen and self-expands, the pre-shaped basket or half spherical shaped frame with covering self-expands within the aneurysm to fill the aneurysm, and the flared-out base with disk self-expands within the artery to cover the narrow to medium neck, blocking the flow of blood into the aneurysm.

The frame 505 has a basket or half spherical shape 515 forming a central opening with a closed end 520. The closed end includes a hub or band 522 to keep the closed end from opening during compression and expansion. In the embodiment shown, the basket or half spherical shape 515 is made of petal shaped arms extending upward and joined at the top. The hub or band 522 is coupled to the closed end 520 to keep the closed end from opening during self-expansion and the flared out base 545 extends outwardly from the hub or band 522.

The frame 505 is be made of a self-expanding material, such as Nitinol, that is laser cut and pre-shaped in the basket or half spherical shape. In other embodiments, the frame 505 is be made of a self-expanding wire material, such as Nitinol wire, that are joined to form the basket or half spherical shape.

The covering 560 is designed to be a barrier that is reinforced by the basket or half spherical shape 515 against the wall of the aneurysm. The covering 560 may be made from a polyurethane and/or ePTFE film. The disk 550 is designed to be a barrier that is reinforced by the flared base 545. The disk 550 may be made from a polyurethane and/or ePTFE film. The disk 550 and/or covering 560 may also include a coating or film can be a platform as a drug carrier promoting the endothelial cell.

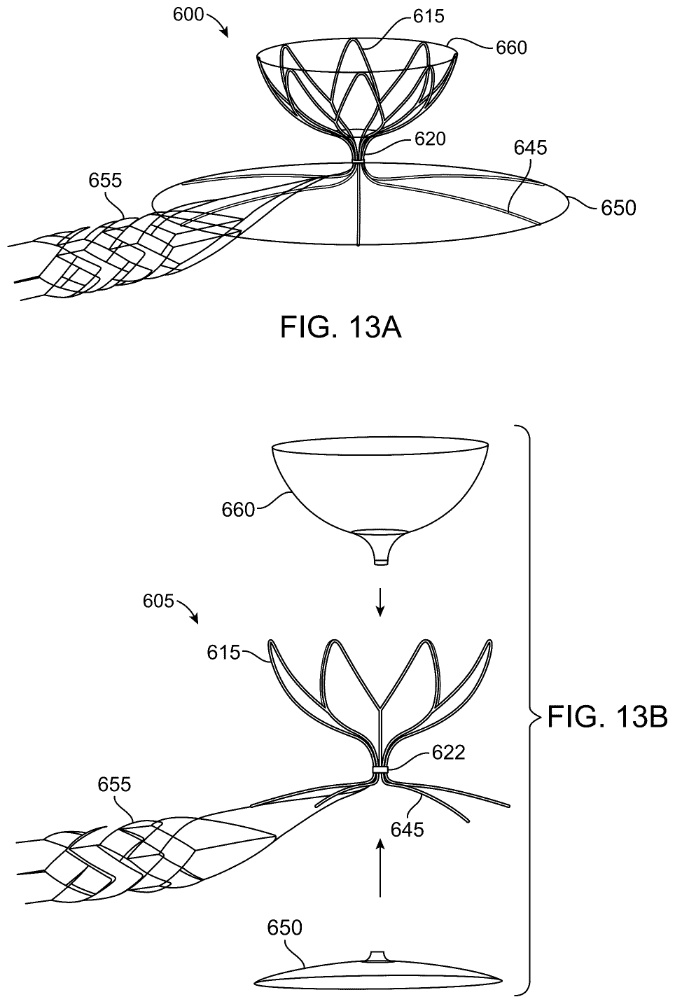

FIGS. 13A, 13B show one embodiment of a cerebral aneurysm device 600 designed to prevent blood from entering a wide neck cerebral aneurysm. The cerebral aneurysm device 600 is similar to cerebral aneurysm device 500 with the addition of a stent base 655. The cerebral aneurysm device 600 is a multiple component device including a frame 605 having basket or half spherical shape 615 with a central opening and a closed end 620, a flared-out base 645 and a stent base 660, a covering 660 coupled to basket or half spherical shape 615, and a disk 650 coupled to the flared-out base 645. The cerebral aneurysm device 600 may be collapsed or compressed into a delivery configuration sized to fit within a lumen of a delivery catheter and delivered to the wide neck cerebral aneurysm. The cerebral aneurysm device 600 exists the lumen and self-expands, the pre-shaped basket or half spherical shaped frame self-expands within the aneurysm to fill the aneurysm, the flared-out base with disk 650 self-expands in the artery to cover the wide neck blocking the flow of blood into the aneurysm, and the stent base 655 self-expands within the artery to anchor the cerebral aneurysm device 600.

The frame 605 has a basket or half spherical shape 615 forming a central opening with a closed end 620, a flared base 645 and a stent base 655. The closed end includes a hub or band 622 to keep the closed end from opening during compression and expansion. The frame 605 may be made of a self-expanding material, such as Nitinol, that is laser cut and pre-shaped.

The covering 660 is designed to be a barrier that is reinforced by the basket or half spherical shape 615 against the wall of the aneurysm. The covering 660 may be made from a polyurethane and/or ePTFE film. The disk 650 is designed to be a barrier that is reinforced by the flared base 645. The disk 650 may be made from a polyurethane and/or ePTFE film. The hub or band 622 is coupled to the closed end 620 to keep the closed end from opening during self-expansion and the flared out base 645 and stent base 655 extend outwardly from the hub or band 622. The disk 650 and/or covering 660 may also include a coating or film can be a platform as a drug carrier promoting the endothelial cell.

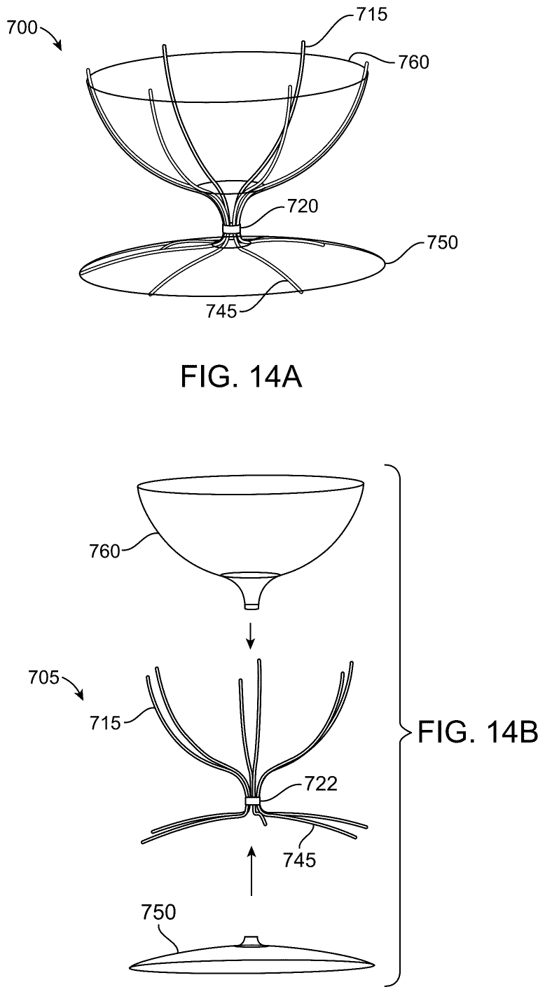

FIGS. 14A, 14B show one embodiment of a cerebral aneurysm device 700 designed to prevent blood from entering a narrow to medium neck cerebral aneurysm. The cerebral aneurysm device 700 is a multiple component device including a frame 705 having basket or half spherical shape 715 and a flared base 745, a covering 760 coupled to basket or half spherical shape 715, and a disk 750 coupled to the flared-out base 745. The cerebral aneurysm device 700 may be may be collapsed or compressed into a delivery configuration sized to fit within a lumen of a delivery catheter and delivered to the narrow to medium neck cerebral aneurysm. The cerebral aneurysm device 700 exits the lumen and self-expands to prevent blood from entering, with the pre-shaped basket or half spherical shaped framed self-expanding within the aneurysm to fill the aneurysm, and the flared-out base with disk self-expanding in the artery to cover the narrow to medium neck, blocking the flow of blood into the aneurysm.

The frame 705 has a basket or half spherical shape 715 forming a central opening with a closed end 720. The closed end includes a hub or band 722 to keep the closed end from opening during compression and expansion. In the embodiment shown, the basket or half spherical shape 715 is made of individual wire or fingers extending upward and joined at the top. The frame 705 is be made of a self-expanding material, such as Nitinol, that is laser cut and pre-shaped in the basket or half spherical shape. In other embodiments, the frame 705 is be made of a self-expanding wire material, such as Nitinol wire, that are joined to form the basket or half spherical shape. The hub or band 722 is coupled to the closed end 720 to keep the closed end from opening during self-expansion and the flared out base 745 extends outwardly from the hub or band 222.

The covering 760 is designed to be a barrier that is reinforced by the basket or half spherical shape 715 against the wall of the aneurysm. The covering 760 may be made from a polyurethane and/or ePTFE film. The disk 750 is designed to be a barrier that is reinforced by the flared base 745. The disk 750 may be made from a polyurethane and/or ePTFE film. The disk 750 and/or covering 760 may also include a coating or film can be a platform as a drug carrier promoting the endothelial cell.

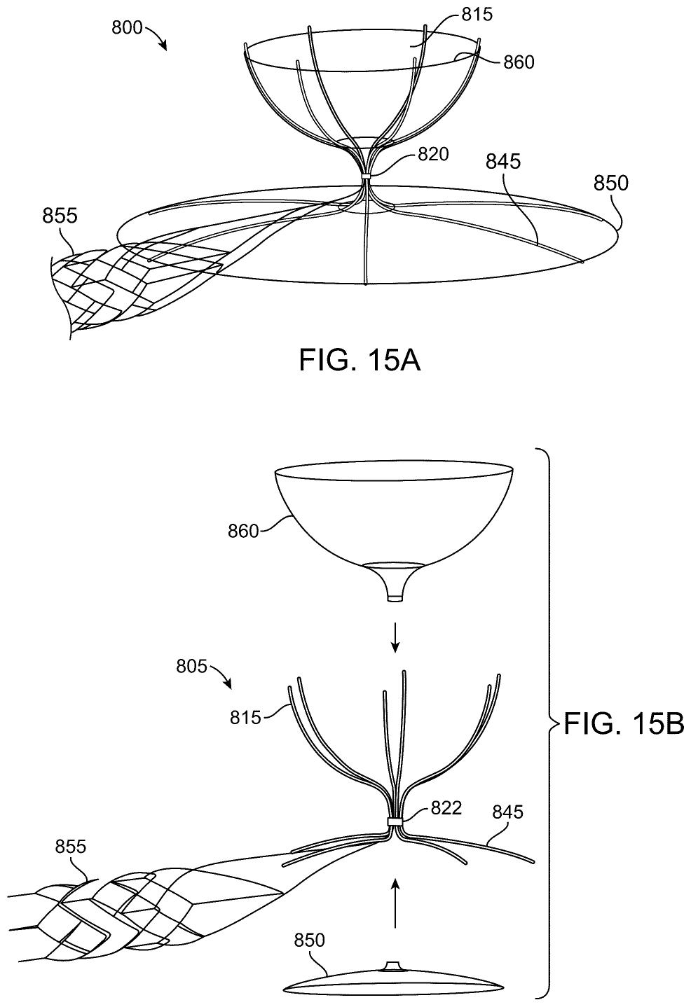

FIGS. 15A, 15B show one embodiment of a cerebral aneurysm device 800 designed to prevent blood from entering a wide neck cerebral aneurysm. The cerebral aneurysm device 800 is similar to cerebral aneurysm device 700 with the addition of a stent base 855. The cerebral aneurysm device 800 is a multiple component device including a frame 805 having basket or half spherical shape 815 with a central opening and a closed end 820, a flared-out base 845 and a stent base 860, a covering 860 coupled to basket or half spherical shape 815, and a disk 850 coupled to the flared-out base 845. The cerebral aneurysm device 800 may be collapsed or compressed into a delivery configuration sized to fit within a lumen of a delivery catheter and delivered to the wide neck cerebral aneurysm. The cerebral aneurysm device 800 exits the lumen and self-expands, with the pre-shaped basket or half spherical shaped frame with covering self-expanding within the aneurysm to fill the aneurysm, the flared-out base with disk 850 self-expanding in the artery to cover the wide neck, blocking the flow of blood into the aneurysm, and the stent base 855 self-expand within the artery to anchor the cerebral aneurysm device 800.

The frame 805 has a basket or half spherical shape 815 forming a central opening with a closed end 820, a flared base 845 and a stent base 855. The closed end includes a hub or band 822 to keep the closed end from opening during compression and expansion and the flared out base 845 and stent 855 extend outwardly from the hub or band 822. The frame 805 may be made of a self-expanding material, such as Nitinol, that is laser cut and pre-shaped.

The covering 860 is designed to be a barrier that is reinforced by the basket or half spherical shape 815 against the wall of the aneurysm. The covering 860 may be made from a polyurethane film. The disk 850 is designed to be a barrier that is reinforced by the flared base 845. The disk 850 may be made from a polyurethane and/or ePTFE film. The disk 850 and/or covering 860 may also include a coating or film can be a platform as a drug carrier promoting the endothelial cell.

A method of placing a cerebral aneurysm device into a cerebral aneurysm includes delivering the cerebral aneurysm device in a compressed configuration within a delivery catheter, inserting the distal end of the delivery catheter through the neck of the cerebral aneurysm, withdrawing the delivery catheter and delivered to self-expand the cerebral aneurysm device within the cerebral aneurysm. In some embodiments, a disk self-expands and covers the neck of the cerebral aneurysm. In some embodiments, a stent base self-expands within the artery to anchor the cerebral aneurysm device within the cerebral aneurysm.

Although the present invention has been illustrated and described herein with reference to some embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A cerebral aneurysm device comprising:
a half spherical shaped frame sized to fit within a cerebral aneurysm having a central opening with an open end and a closed end;
a hub or band coupled to the closed end to keep the closed end from opening;
a spherical-shaped filler designed to fit within the half spherical shaped frame; and
a coating or film configured to cover and seal the spherical-shaped filler;
wherein the half spherical shaped frame is positioned within a cerebral aneurysm with the closed end positioned in a neck of the cerebral aneurysm to seal the neck and prevent blood from entering the cerebral aneurysm.

2. The cerebral aneurysm device of claim 1, wherein the half spherical shaped frame and the spherical-shaped filler are made of a self-expanding material.

3. The cerebral aneurysm device of claim 2, wherein the self-expanding half spherical shaped frame and spherical-shaped filler be collapsed or compressed into a delivery configuration sized to fit within a lumen of a delivery catheter and delivered to the cerebral aneurysm, exiting the lumen and self-expanding into an expanded configuration, blocking the flow of blood into the aneurysm.

4. The cerebral aneurysm device of claim 2, wherein the spherical-shaped filler includes an inner spherical-shaped coiled portion having a first pitch and first amplitude, an outer spherical-shaped coil portion having a second pitch and second amplitude.

5. The cerebral aneurysm device of claim 1, wherein the half spherical shaped frame includes arms extending upward from the closed end.

6. The cerebral aneurysm device of claim 1, wherein the spherical-shaped filler includes a segment configured to attach to the closed end.

7. The cerebral aneurysm device of claim 1, wherein the coating or film is made from a polyurethane, ePTFE and/or other collagen film.

8. The cerebral aneurysm device of claim 1, wherein the coating or film is a platform as a drug carrier.

9. The cerebral aneurysm device of claim 1, wherein the half spherical shaped frame includes a flared-out base coupled with the hub or band with a disk coating couple to the flared-out base.

10. The cerebral aneurysm device of claim 1, further comprising a self-expanding stent coupled with the hub or band.

11. A cerebral aneurysm device comprising:
a half spherical shaped frame sized to fit within a cerebral aneurysm with an open end and a closed end;
a hub or band coupled to the closed end;
a flared-out base coupled to the hub or band extending outward;
a covering coupled to an outer surface of the half spherical shaped frame; and
a disk coating coupled to the flared-out base;
wherein the half spherical shaped frame is positioned within a cerebral aneurysm with the closed end positioned in a neck of the cerebral aneurysm and the flared-out base with disk coating extend from the other side of the neck to seal the neck and prevent blood from entering the cerebral aneurysm.

12. The cerebral aneurysm device of claim 11, wherein the half spherical shaped frame and flared-out base are made of a self-expanding material.

13. The cerebral aneurysm device of claim 12, wherein the half spherical shaped frame with covering and the flared-out base with disk coating may be collapsed or compressed into a delivery configuration sized to fit within a lumen of a delivery catheter and delivered to the cerebral aneurysm, exiting the lumen and self-expanding into an expanded configuration, blocking the flow of blood into the aneurysm.

14. The cerebral aneurysm device of claim 11, wherein the half spherical shape includes arms extending upward from the closed end.

15. The cerebral aneurysm device of claim 11, wherein the covering and/or disk coating are made from a polyurethane, ePTFE and/or other collagen film.

16. The cerebral aneurysm device of claim 11, wherein the covering and/or disk coating are a platform as a drug carrier.

17. The cerebral aneurysm device of claim 11, further comprising a self-expanding stent coupled with the hub or band.

18. A cerebral aneurysm device comprising:
a self-expanding half spherical shaped frame with an outer covering;
a hub or band coupled to a closed end of the half spherical shaped frame; and
a self-expanding flared-out base with a disk coating extending outward from the hub or band;
wherein the half spherical shaped frame with covering and the flared-out base with disk coating may be collapsed or compressed into a delivery configuration sized to fit within a lumen of a delivery catheter and delivered to the cerebral aneurysm, exiting the lumen and self-expanding into an expanded configuration, blocking the flow of blood into the aneurysm.

19. The cerebral aneurysm device of claim 18, wherein the covering and/or disk coating are made from a polyurethane, ePTFE and/or other collagen film.

20. The cerebral aneurysm device of claim 18, wherein the covering and/or disk coating are a platform as a drug carrier.
